SEQUENCE LISTING (8 sequences)

SEQ ID NO: 1 - DNA, 21 bp, Artificial Sequence
  Initiator for nucleic acid synthesis
cagggatccgtgaagctatcc

SEQ ID NO: 2 - DNA, 24 bp, Artificial Sequence
  Product synthesized de novo
cagggatccgtgaagctatccuat

SEQ ID NO: 3 - DNA, 16 bp, Artificial Sequence
  Synthesized nucleic acid
gcgtctaggactaagc

SEQ ID NO: 4 - DNA, 38 bp, Artificial Sequence
  Nucleic acid containing an initiator, a linking dUMP, and a newly synthesized 16-mer polynucleotide
cagggatccgtgaagctatccugcgtctaggactaagc

SEQ ID NO: 5 - DNA, 24 bp, Artificial Sequence
  Product synthesized de novo
  misc_feature (22)..(22): n is inosine
cagggatccgtgaagctatccnat

SEQ ID NO: 6 - DNA, 38 bp, Artificial Sequence
  Nucleic acid containing an initiator, a linking dIMP, and a newly synthesized 16-mer polynucleotide
  misc_feature (22)..(22): n is inosine
cagggatccgtgaagctatccngcgtctaggactaagc

SEQ ID NO: 7 - DNA, 38 bp, Artificial Sequence
  Template
gcttagtcctagacgcaggatagcttcacggatccctg

SEQ ID NO: 8 - protein, 775 aa, Artificial Sequence
  Pfu(exo-) DNA polymerase
MILDVDYITEEGKPVIRLFKKENGKFKIEHDRTFRPYIYALLRDDSKIEEVKKITGERHGKIVRIVDVEKVEKKFLGKPITVWKLYLEHPQDVPTIREKVREHPAVVDIFEYDIPFAKRYLIDKGLIPMEGEEELKILAFAIATLYHEGEEFGKGPIIMISYADENEAKVITWKNIDLPYVEVVSSEREMIKRFLRIIREKDPDIIVTYNGDSFDFPYLAKRAEKLGIKLTIGRDGSEPKMQRIGDMTAVEVKGRIHFDLYHVITRTINLPTYTLEAVYEAIFGKPKEKVYADEIAKAWESGENLERVAKYSMEDAKATYELGKEFLPMEIQLSRLVGQPLWDVSRSSTGNLVEWFLLRKAYERNEVAPNKPSEEEYQRRLRESYTGGFVKEPEKGLWENIVYLDFRALYPSIIITHNVSPDTLNLEGCKNYDIAPQVGHKFCKDIPGFIPSLLGHLLEERQKIKTKMKETQDPIEKILLDYRQKAIKLLANSFYGYYGYAKARWYCKECAESVTAWGRKYIELVWKELEEKFGFKVLYIDTDGLYATIPGGESEEIKKKALEFVKYINSKLPGLLELEYEGFYKRGFFVTKKRYAVIDEEGKVITRGLEIVRRDWSEIAKETQARVLETILKHGDVEEAVRIVKEVIQKLANYEIPPEKLAIYEQITRPLHEYKAIGPHVAVAKKLAAKGVKIKPGMVIGYIVLRGDGPISNRAILAEEYDPKKHKYDAEYYIENQVLPAVLRILEGFGYRKEDLRYQKTRQVGLTSWLNIKKS

(19) United States
(12) Patent Application Publication
(10) Pub. No.: US 2022/0195476 A1
(43) Pub. Date: Jun. 23, 2022

(54) METHOD AND KIT FOR REGENERATING REUSABLE INITIATORS FOR NUCLEIC ACID SYNTHESIS

(71) Applicant: Cheng-Yao CHEN, Hsinchu (TW)

(72) Inventors: Cheng-Yao CHEN, Hsinchu (TW); Yi-Wen CHENG, Kaohsiung City (TW)

(21) Appl. No.: 17/128,677

(22) Filed: Dec. 21, 2020

(51) Int. Cl. C12P 19/34

(52) U.S. Cl. CPC C12P 19/34

(57) ABSTRACT

A method for nucleic acid synthesis and regeneration of a reusable synthesis initiator includes incorporating a linking nucleotide to an immobilized initiator using a polymerase, synthesizing a nucleic acid right after the linking nucleotide using the polymerase, subjecting a substrate base of the linking nucleotide in the nucleic acid to base-excision by a DNA glycosylase to generate an abasic site, subjecting the abasic site to cleavage by an endonuclease to release the nucleic acid from the initiator, and converting the 3′ terminus of the initiator back to its original form by a 3′ phosphatase activity-possessing enzyme. A kit based on the aforesaid method and a method for regenerating a reusable initiator are also disclosed.

### FIELD

The disclosure relates to a method and a kit for regenerating reusable initiators for nucleic acid synthesis by virtue of enzymes.

### BACKGROUND

DNA synthesis methods, including template-dependent and template-independent DNA synthesis methods, require an initiator (i.e. a short polynucleotide) that serves as a primer for nucleotide additions. However, after DNA synthesis, such an initiator is normally not reusable and discarded. Therefore, a new initiator is required for each round of new DNA synthesis, increasing the overall production cost thereof and rendering such synthesis inconvenient.

To facilitate the cost-efficient and robust DNA synthesis process, there is a need to develop a novel approach to synthesize DNA and render a reusable initiator for new synthesis.

### SUMMARY

Therefore, an object of the disclosure is to provide a method and a kit for nucleic acid synthesis and regeneration of a reusable initiator for such synthesis, which can alleviate at least one of the drawbacks of the prior art.

Such method includes:
exposing an initiator attached to a solid support for nucleic acid synthesis to a linking nucleotide in the presence of a polymerase so that the linking nucleotide is incorporated to the initiator, the linking nucleotide having a substrate base, a substrate sugar, and a 3′ hydroxyl group;
exposing the initiator containing the linking nucleotide to nucleotide monomers in the presence of the polymerase, so that a nucleic acid is synthesized and is coupled to the initiator right after the linking nucleotide;
providing a mono-functional DNA glycosylase, the linking nucleotide with the substrate base being recognizable and excisable by the mono-functional DNA glycosylase;
subjecting the substrate base to an excision treatment with the mono-functional DNA glycosylase, so that the substrate base is excised by the mono-functional DNA glycosylase to generate an abasic site;
providing an abasic site endonuclease, the resulting abasic site being recognizable and the substrate sugar being cleavable by the abasic site endonuclease;
subjecting the abasic site to a cleavage treatment with the abasic site endonuclease, so that the substrate sugar and the backbone of the nucleic acid at the abasic site are both cleaved to release the newly synthesized nucleic acid from the initiator, so that a 3′-terminal nucleotide of the initiator leaves a 3′ phosphate group, and so that a 5′-terminal nucleotide of the newly synthesized nucleic acid has a 5′ phosphate group;
providing a 3′ phosphatase activity-possessing enzyme; and
subjecting the 3′-terminal nucleotide of the initiator to a dephosphorylation treatment with the 3′ phosphatase activity-possessing enzyme, so that the 3′ phosphate group of the 3′-terminal nucleotide of the initiator is converted back to the original 3′ hydroxyl group for the initiator to be reusable for a new round of synthesis reaction.

The kit includes a polymerase, a linking nucleotide, a mono-functional DNA glycosylase, an abasic site endonuclease, and a3′ phosphatase activity-possessing enzyme. The kit is used according to the aforesaid method.

Another object of the disclosure is to provide a method of regenerating a reusable initiator for nucleic acid synthesis, which can alleviate at least one of the drawbacks of the prior art.

Such method includes:
providing a mono-functional DNA glycosylase;
providing an initiator and a newly synthesized nucleic acid, the initiator being linked to a solid support, the newly synthesized nucleic acid being linked to the initiator right after a linking nucleotide having a substrate base and a substrate sugar, the linking nucleotide with the substrate base being recognizable and excisable by the mono-functional DNA glycosylase;
subjecting the substrate base to an excision treatment with the mono-functional DNA glycosylase, so that the substrate base is excised by the mono-functional DNA glycosylase to generate an abasic site;
providing an abasic site endonuclease, the resulting abasic site being recognizable and the substrate sugar being cleavable by the abasic site endonuclease;
subjecting the abasic site to a cleavage treatment with the abasic site endonuclease, so that the substrate sugar and the backbone of the nucleic acid at the abasic site are both cleaved to release the newly synthesized nucleic acid from the initiator, so that a 3′-terminal nucleotide of the initiator leaves a 3′ phosphate group, and so that a 5′-terminal nucleotide of the newly synthesized nucleic acid has a 5′ phosphate group;
providing a 3′ phosphatase activity-possessing enzyme; and
subjecting the 3′-terminal nucleotide of the initiator to a dephosphorylation treatment with the 3′ phosphatase activity-possessing enzyme, so that the 3′ phosphate group of the 3′-terminal nucleotide of the initiator is converted back to an original 3′ hydroxyl group for the initiator to be reusable for a new round of synthesis reaction.

### BRIEF DESCRIPTION OF THE DRAWINGS

Other features and advantages of the disclosure will become apparent in the following detailed description of the embodiments with reference to the accompanying drawings, of which:

FIG. 1 is a schematic diagram illustrating template-independent nucleic acid synthesis and reversion of an initiator back to its original form as applied in Example 1, infra, in which the symbol “U” represents a linking deoxyuridine, the symbol “N” represents an incorporated nucleoside monomer, the symbol “UDG” represents uracil-DNA glycosylase, the symbol “Nei” represents endonuclease VIII, and the symbol “T4 PNKP” represents T4 polynucleotide kinase with 3′ phosphatase activity;

FIG. 2 is a fluorescent image of urea-polyacrylamide gel showing the feasibility of template-independent nucleic acid synthesis validated in section 1 of Example 1, infra;

FIG. 3 is a fluorescent image of urea-polyacrylamide gel showing results of Example 1, infra, in which the symbol “S” represents a polynucleotide containing an initiator and a newly synthesized nucleic acid with a linking deoxyuridine, the symbol “U” represents a treatment with UDG only, the symbol “N” represents a treatment with Nei only, the symbol “U+N” represents treatments with UDG and Nei, and the symbol “U+N+P” represents treatments with UDG, Nei, and T4 PNKP;

FIG. 4 is a schematic diagram illustrating template-independent nucleic acid synthesis and reversion of an initiator to its original form as applied in Example 2, infra, in which the symbol “I” represents a linking deoxyinosine, the symbol “N” represents an incorporated nucleoside monomer, the symbol “AAG” represents alkyladenine DNA glycosylase, the symbol “Nei” represents endonuclease VIII, and the symbol “T4 PNKP” represents T4 polynucleotide kinase with 3′ phosphatase activity;

FIG. 5 is a fluorescent image of urea-polyacrylamide gel showing the feasibility of template-independent nucleic acid synthesis validated in section 1 of Example 2, infra;

FIG. 6 is a fluorescent image of urea-polyacrylamide gel showing results of Example 2, infra, in which the symbol “S” represents a polynucleotide containing an initiator and a newly synthesized nucleic acid with a linking deoxyinosine, the symbol “A” represents a treatment with AAG only, the symbol “N” represents a treatment with Nei only, the symbol “A+N” represents treatments with AAG and Nei, and the symbol “A+N+P” represents treatments with AAG, Nei, and T4 PNKP;

FIG. 7 is a schematic diagram illustrating template-dependent nucleic acid synthesis and reversion of an initiator to its original form as applied in Example 3, infra, in which the symbol “U” represents a linking deoxyuridine, the symbol “N” represents a nucleoside, the symbol “UDG” represents uracil-DNA glycosylase, the symbol “Nei” represents endonuclease VIII, and the symbol “T4 PNKP” represents T4 polynucleotide kinase with 3′ phosphatase activity;

FIG. 8 is a fluorescent image of urea-polyacrylamide gel showing results of Example 3, infra, in which the symbol “S” represents a duplex polynucleotide containing an initiator and a newly synthesized nucleic acid with a linking deoxyuridine, the symbol “U” represents a treatment with UDG only, the symbol “N” represents a treatment with Nei only, the symbol “U+N” represents treatments with UDG and Nei, and the symbol “U+N+P” represents treatments with UDG, Nei, and T4 PNKP;

FIG. 9 is a schematic diagram illustrating template-dependent nucleic acid synthesis and reversion of an initiator to its original form as applied in Example 4, infra, in which the symbol “I” represents a linking deoxyinosine, the symbol “N” represents an incorporated nucleoside monomer, the symbol “AAG” represents alkyladenine DNA glycosylase, the symbol “Nei” represents endonuclease VIII, and the symbol “T4 PNKP” represents T4 polynucleotide kinase with 3′ phosphatase activity; and

FIG. 10 is a fluorescent image of urea-polyacrylamide gel showing results of Example 4, infra, in which the symbol “S” represents a duplex polynucleotide containing an initiator and a newly synthesized nucleic acid with a linking deoxyinosine, the symbol “A” represents a treatment with AAG only, the symbol “N” represents a treatment with Nei only, the symbol “A+N” represents treatments with AAG and Nei, and the symbol “A+N+P” represents treatments with AAG, Nei, and T4 PNKP.

### DETAILED DESCRIPTION

It is to be understood that, if any prior art publication is referred to herein, such reference does not constitute an admission that the publication forms a part of the common general knowledge in the art, in Taiwan or any other country.

For the purpose of this specification, it will be clearly understood that the word “comprising” means “including but not limited to”, and that the word “comprises” has a corresponding meaning.

Unless defined otherwise, all technical and scientific terms used herein have the meaning commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described.

The present disclosure provides a method for nucleic acid synthesis and regeneration of a reusable initiator for such synthesis, which includes:
exposing an initiator attached to a solid support for nucleic acid synthesis to a linking nucleotide in the presence of a polymerase so that the linking nucleotide is incorporated to the initiator, the linking nucleotide having a substrate base, a substrate sugar, and a 3′ hydroxyl group,
exposing the initiator containing the linking nucleotide to nucleotide monomers in the presence of the polymerase, so that a nucleic acid is synthesized and is coupled to the initiator right after the linking nucleotide;
providing a mono-functional DNA glycosylase, the linking nucleotide with the substrate base being recognizable and excisable by the mono-functional DNA glycosylase;
subjecting the substrate base to an excision treatment with the mono-functional DNA glycosylase, so that the substrate base is excised from the linking nucleotide by the mono-functional DNA glycosylase to generate an abasic site;
providing an abasic site endonuclease, the resulting abasic site being recognizable and the substrate sugar being cleavable by the abasic site endonuclease;
subjecting the abasic site to a cleavage treatment with the abasic site endonuclease, so that the substrate sugar and the backbone of the nucleic acid at the abasic site are both cleaved to release the newly synthesized nucleic acid from the initiator, so that a 3′-terminal nucleotide of the initiator leaves a 3′ phosphate group, and so that a 5′-terminal nucleotide of the synthesized nucleic acid has a 5′ phosphate group;
providing a 3′ phosphatase activity-possessing enzyme; and
subjecting the 3′-terminal nucleotide of the initiator to a dephosphorylation treatment with the 3′ phosphatase activity-possessing enzyme, so that the 3′ phosphate group of the 3′-terminal nucleotide of the initiator is converted back to the original 3′ hydroxyl group for the initiator to be reusable for a new round of synthesis reaction.

According to the present disclosure, the excision treatment, the cleavage treatment, and the dephosphorylation treatment may be conducted simultaneously or sequentially.

The terms “nucleic acid”, “nucleic acid sequence”, and “nucleic acid fragment” as used herein refer to a deoxyribonucleotide or ribonucleotide sequence in single-stranded or double-stranded form, and comprise naturally occurring nucleotides or artificial chemical mimics. The term “nucleic acid” as used herein is interchangeable with the terms “oligonucleotide”, “polynucleotide”, “gene”, “DNA”, “cDNA”, “RNA”, and “mRNA” in use.

The term “initiator” refers to a mononucleoside, a mononucleotide, an oligonucleotide, a polynucleotide, or modified analogs thereof, from which a nucleic acid is to be synthesized. The term “initiator” may also refer to a Xeno nucleic acid (XNA) or a peptide nucleic acid (PNA) haying a 3′-hydroxyl group.

According to the present disclosure, the initiator may be in a template-independent form or a template-dependent form (namely, the initiator may not be annealed or hybridized to a complementary template, or may be annealed to a template to form a duplex or a double strand).

When the initiator is in a template-independent form, the initiator may have a sequence selected from a non-self complementary sequence and a non-self complementarity forming sequence. The term “self complementary” means that a sequence (e.g. a nucleotide sequence, a XNA, or a PNA sequence) folds back on itself (i.e. a region of the sequence binds or hybridizes to another region of the sequence), creating a duplex, double-strand like structure which can serve as a template for nucleic acid synthesis. Depending on how close together the complementary regions of the sequence are, the strand may form, for instance, hairpin loops, junctions, bulges or internal loops. The term “self complementarity forming” is used to describe a sequence (e.g. a nucleotide sequence, a XNA, or a PNA sequence) from which a complementary extended portion is formed when such sequence serves as a template (namely, a self-complementary sequence is formed based on such sequence serving as a template). For instance, the self complementarity forming sequence maybe “ATCC”. When the “ATCC” sequence serves as a template, an extended portion “GGAT” complementary to such sequence is formed from such sequence (i.e. a self-complementary sequence “ATCCGGAT” is formed).

Generally, a “template” is a polynucleotide that contains the target nucleotide sequence. In some instances, the terms “target sequence”, “template polynucleotide”, “target nucleic acid”, “target polynucleotide”, “nucleic acid template”, “template sequence”, and variations thereof, are used interchangeably. Specifically, the term “template” refers to a strand of nucleic acid on which a complementary copy is synthesized from nucleotides or nucleotide analogs through the activity of a template-dependent nucleic acid polymerase. Within a duplex, the template strand is, by convention, depicted and described as the “bottom” strand. Similarly, the non-template strand is often depicted and described as the “top” strand. The “template” strand may also be referred to as the “sense” strand, and the non-template strand as the “antisense” strand.

According to the present disclosure, the initiator has a 5′ end linked to the solid support, and the linking nucleotide is coupled to a 3′-terminal nucleotide of the initiator and a 5′-terminal nucleotide of the synthesized nucleic acid. The initiator may be directly attached to the support, or may be attached to the support via a linker.

Examples of the solid support include, but are not limited to, microarrays, beads (coated or non-coated), columns, optical fibers, wipes, nitrocellulose, nylon, glass, quartz, diazotized membranes (paper or nylon), silicones, polyformaldehyde, cellulose, cellulose acetate, paper, ceramics, metals, metalloids, semiconductive materials, magnetic particles, plastics (such as polyethylene, polypropylene, and polystyrene, gel-forming materials [such as proteins (e.g., gelatins), lipopolysaccharides, silicates, agarose, polyacrylamides, methylmethracrylate polymers], sol gels, porous polymer hydrogels, nanostructured surfaces, nanotubes (such as carbon nanotubes), and nanoparticles (such as gold nanoparticles or quantum dots).

According to the present disclosure, depending on the form of the initiator, the synthesized nucleic acid and the linking nucleotide may each be in a template-independent form or a template-dependent form.

As used herein, the term “incorporated” or “incorporation” refers to becoming a part of a nucleic acid. There is a known flexibility in the terminology regarding incorporation of nucleic acid precursors. For example, the nucleotide dGTP is a deoxyribonucleoside triphosphate. Upon incorporation into DNA, dGTP becomes dGMP, that is, a deoxyguanosine monophosphate moiety. Although DNA does not include dGTP molecules, one may say that one incorporates dGTP into DNA.

According to the present disclosure, the nucleotide monomers may be a natural nucleic acid nucleotide whose constituent elements are a sugar, a phosphate group and a nitrogen base. The sugar may be ribose in RNA or 2′-deoxyribose in DNA. Depending on whether the nucleic acid to be synthesized is DNA or RNA, the nitrogen base is selected from adenine, guanine, uracil, cytosine and thymine. Alternatively, the nucleotide monomers may be a nucleotide which is modified in at least one of the three constituent elements. By way of example, the modification can take place at the level of the base, generating a modified product (such as inosine, methyl-5-deoxycytidine, deoxyuridine, dimethylamino-5-deoxyuridine, diamino-2,6-purine or bromo-5-deoxyuridine, and any other modified base which permits hybridization), at the level of the sugar (for example, replacement of a deoxyribose by an analog), or at the level of the phosphate group (for example, boronate, alkylphosphonate, or phosphorothioate derivatives).

According to the present disclosure, the nucleotide monomer may have a removable blocking moiety. Examples of the removable blocking moiety include, but are not limited to, a 3′-O-blocking moiety, a base blocking moiety, and a combination thereof.

The nucleotide monomer having a removable blocking moiety is also referred to as a reversible terminator. Therefore, the nucleotide monomer having the 3′-O-blocking moiety is also referred to as 3′-blocked reversible terminator or a 3′-O-modified reversible terminator, and the nucleotide monomer having a base blocking moiety is also referred to as a 3′-unblocked reversible terminator or a 3′-OH unblocked reversible terminator.

As used herein, the term “reversible terminator” refers to a chemically modified nucleotide monomer. When such a reversible terminator is incorporated into a growing nucleic acid by a polymerase, it blocks the further incorporation of another nucleotide monomer by the polymerase. Such “reversible terminator” base and a nucleic acid can be deprotected by chemical or physical treatment, and following such deprotection, the nucleic acid can be further extended by a polymerase.

Examples of the 3′-O-blocking moiety include, but are not limited to, O-azidomethyl, O-amino, O-allyl, O-phenoxyacetyl, O-methoxyacetyl, O-acetyl, O-(p-toluene)sulfonate, O-phosphate, O-nitrate, O-[4-methoxy]-tetrahydrothiopyranyl, O-tetrahydrothiopyranyl, O-[5-methyl]-tetrahydrofuranyl, O-[2-methyl,4-methoxy]-tetrahydropyranyl, O-[5-methyl]-tetrahydropyranyl, and O-tetrahydrothiofuranyl, 0-2-nitrobenzyl, 0-methyl, and O-acyl.

Examples of the 3′-unblocked reversible terminators include, but are not limited to, 7-[(S)-1-(5-methoxy-2-nitrophenyl)-2,2-dimethyl-propyloxy]methyl-7-deaza-dATP, 5-[(S)-1-(5-methoxy-2-nitrophenyl) -2,2-dimethyl-propyloxy]methyl-dCTP, 1-(5-methoxy-2-nitrophenyl)-2,2-dimethyl-propyloxy]methyl-7-deaza-dGTP, 5-[(S)-1-(5-methoxy-2-nitrophen-yl)-2,2-dimethyl-propyloxy]methyl-dUTP, and 5-[(S)-1-(2-nitrophenyl)-2,2-dimethyl-propyloxy]methyl-dUTP.

According to the present disclosure, the base blocking moiety may be a reversible dye-terminator. Examples of the reversible dye-terminator include, but are not limited to, a reversible dye-terminator of Illumina NovaSeq, a reversible dye-terminator of Illumina NextSeq, a reversible dye-terminator of Illumina MiSeq, a reversible dye-terminator of Illumina HiSeq, a reversible dye-terminator of Illumina Genome Analyzer IIX, a lightning terminator of LaserGen, and a reversible dye-terminator of Helicos Biosciences Heliscope.

Since the reversible terminators are well-known to and commonly used by those skilled in the art, further details of the same are omitted herein for the sake of brevity. Nevertheless, applicable 3′-blocked reversible terminators, applicable 3′-unblocked reversible terminators, and applicable conditions for protection and deprotection (i.e. conditions for adding and eliminating the removable blocking moiety) can be found in, for example, Gardner et al. (2012), Nucleic Acids Research, 40(15):7404-7415, Litosh et al. (2011), Nucleic Acids Research, 39(6):e39, and Chen et al. (2013), Genomics Proteomics Bioinformatics, 11:34-40.

According to the present disclosure, the polymerase may be a template-dependent polymerase or a template-independent polymerase.

According to the present disclosure, the polymerase may be selected from the group consisting of a family-A DNA polymerase (e.g. T7 DNA polymerase, Pol I, Pol γ, θ, and ν), a family-B DNA polymerase (e.g. Pol II, Pol B, Pol ζ, Pol α, δ, and ε), a family-C DNA polymerase (e.g. Pol III), a family-D DNA polymerase (e.g. PolD), a family-X DNA polymerase (e.g. Pol β, Pol σ, Pol λ, Pol μ, and terminal deoxynucleotidyl transferase), a family-Y DNA polymerase (e.g. Pol ι, Pol κ, Pol η, DinB, Pol IV, and Pol V), a reverse transcriptase (e.g. telomerase and hepatitis B virus), and enzymatically active fragments thereof.

Non-limiting examples of widely employed template-dependent polymerases include T7 DNA polymerase of the phage T7 and T3 DNA polymerase of the phage T3 which are DNA-dependent DNA polymerases, T7 RNA polymerase of the phage T7 and T3 RNA polymerase of the phage T3 which are DNA-dependent RNA polymerases, DNA polymerase I or its fragment known as Klenow fragment of Escherichia coli which is a DNA-dependent DNA polymerase, Thermophilus aquaticus DNA polymerase, Tth DNA polymerase and vent DNA polymerase, which are thermostable DNA-dependent DNA polymerases, eukaryotic DNA polymerase β, which is a DNA-dependent DNA polymerase, telomerase which is a RNA-dependent DNA polymerase, and non-protein catalytic molecules such as modified RNA (ribozymes; Unrau & Bartel, 1998) and DNA with template-dependent polymerase activity.

Non-limiting examples of the template-independent polymerases include reverse transcriptases, poly(A) polymerase, DNA polymerase theta (θ), DNA polymerase mu (μ), and terminal deoxynucleotidyl transferase.

Since polymerases suitable for nucleic acid synthesis, linking nucleotide addition, and nucleic acid synthesis are within the expertise and routine skills of those skilled in the art, further details thereof are omitted herein for the sake of brevity.

As used herein, the term “mono-functional DNA glycosylase” refers to naturally existing mono-functional glycosylases that originally have only glycosylase activity. The term “mono-functional DNA glycosylase” also refers to mono-functional glycosylases that are derived from bi-functional DNA glycosylases originally having glycosylase activity and abasic-site lyase activity by removing or inactivating the abasic-site lyase domain of the bi-functional DNA glycosylases.

According to the present disclosure, the mono-functional DNA glycosylase may be selected from the group consisting of uracil-DNA glycosylase (UDG or UNG), alkyladenine DNA glycosylase (AAG; also referred to as methylpurine DNA glycosylase (MPG)), single-strand-selective monofunctional uracil DNA glycosylase 1 (SMUG1), methyl-binding domain glycosylase 4 (MBD4), thymine DNA glycosylase (TDG), mutY homolog DNA glycosylase (MYH), alkylpurine glycosylase C (AlkC), alkylpurine glycosylase D (AlkD), 8-oxo-guanine glycosylase 1 (OGG1) without the abasic site lyase activity, endonuclease III-like 1 (NTHL1) without the abasic site lyase activity, endonuclease VIII-like glycosylase 1 (NEIL1) without the abasic site lyase activity, endonuclease VIII-like glycosylase 2 (NEIL2) without the abasic site lyase activity, endonuclease VIII-like glycosylase 3 (NEIL3) without the abasic site lyase activity, and enzymatically active fragments thereof.

Since removing or inactivating the abasic-site lyase domain of bi-functional DNA glycosylases to obtain mono-functional glycosylases are within the expertise and routine skill of those skilled in the art, details thereof are omitted herein for the sake of brevity.

As used herein, the term “enzymatically active fragment” refers to a fragment of a catalytically or enzymatically active protein or polypeptide which contains at least 10%, preferably at least 20%, even more preferably at least 30%, even more preferably at least 40%, even more preferably at least 50%, even more preferably at least 60%, even more preferably at least 70%, even more preferably at least 80%, even more preferably at least 90%, or even more preferably at least 95% of activity of the protein or polypeptide from which the fragment is derived.

In an exemplary embodiment of the present disclosure, the mono-functional DNA glycosylase is uracil-DNA glycosylase. In another exemplary embodiment of the present disclosure, the mono-functional DNA glycosylase is alkyladenine DNA glycosylase.

The terms “abasic”, “apurinic/apyrimidinic”, and D-spacer can be interchangeably used to indicate a site at which the base is not present, but the sugar phosphate backbone remains intact. Therefore, the abasic site endonuclease is also known as apurinic/apyrimidinic site endonuclease.

According to the present disclosure, the abasic site endonuclease may be selected from the group consisting of endonuclease VIII (Nei), endonuclease III (EndoIII or Nth), and enzymatically active fragments thereof. In an exemplary embodiment, the abasic site endonuclease is endonuclease VIII.

According to the present disclosure, the 3′ phosphatase activity-possessing enzyme may be selected from the group consisting of a polynucleotide kinase 3′-phosphatase, a 3′-phosphoesterase, and enzymatically active fragments thereof. The 3′ phosphatase activity-possessing enzyme may be T4 polynucleotide kinase (PNK) with 3′ phosphatase activity (also referred to as T4 polynucleotide kinase/phosphatase (T4 PNKP)), as well as zinc finger DNA 3′-phosphoesterase (ZDP).

Since the applicable 3′ phosphatase activity-possessing enzymes are within the expertise and routine skills of those skilled in the art, further details thereof are omitted herein for the sake of brevity. Nevertheless, the applicable 3′ phosphatase activity-possessing enzymes can be found in, for instance, Blondal et al. (2005), J. Bio. Chem., 280(7):5188-5194, Dobson et al. (2006), Nucleic Acids Research, 34(8):2230-2237, Blasius et al. (2007), BMC Molecular Biology, 8:69, Coquelle et al. (2011), PNAS, 108(52):21022-21027, Vance et al. (2001), J. Bio. Chem., 276(18):15703-15781, and the NCBI website (https://www.ncbi.nlm.nih.gov/gene?Db=gene&Cmd=Deta ilsSearch&Term=11284#general-protein-info).

According to the present disclosure, the substrate base of the linking nucleotide coupled to the initiator may be selected from the group consisting of uracil, hypoxanthine, thymine, cytosine, guanine, 5-fluorouracil, 5-hydroxymethyluracil, 5-formylcytosine, 5-carboxylcytosine, 3-methyladenine, 3-methylguanine, 7-methyladenine, 7-methylguanine, N⁶-methyladenine, 8-oxo-7,8-dihydroguanine, 5-hydroxylcytosine, 5-hydroxyluracil, dihydroxyuracil, ethenocytosine, ethenoadenine, thymine glycol, cytosine glycol, 2,6-diamino-4-hydroxy-5-N-methylformamidopyrimidine, a formamidopyrimidine derivative of adenine, a formamidopyrimidine derivative of guanine, adenine opposite guanine, uracil opposite guanine, uracil opposite adenine, thymine opposite guanine, ethenocytosine opposite guanine, adenine opposite 8-oxo-7,8-dihydroguanine, and 2-hydroxyladenine opposite guanine. In an exemplary embodiment of the present disclosure, the substrate base of the linking nucleotide is uracil. In another exemplary embodiment of the present disclosure, the substrate base of the linking nucleotide is hypoxanthine.

Since the suitable mono-functional DNA glycosylases and their corresponding substrate bases are within the expertise and routine skill of those skilled in the art, details thereof are omitted herein for the sake of brevity. Nevertheless, the suitable mono-functional DNA glycosylases and their corresponding substrate bases can be found in, for example, Jacobs et al. (2012), Chromosoma, 121:1-20, Krokan et al. (1997), Biochem. J., 325:1-16, and Kim et al. (2012), Current Molecular Pharmacology, 5:3-13.

The term “linking nucleotide” refers to the first nucleotide that is incorporated to the initiator with respect to the newly synthesized nucleic acid.

The term “substrate base” refers to the base of a linking nucleotide that serves as a substrate for an enzyme. The term “substrate sugar” refers to a nucleoside sugar moiety of the linking nucleotide that serves as a substrate for an enzyme.

Furthermore, the present disclosure provides a kit for nucleic acid synthesis and regeneration of a reusable initiator for such synthesis, which includes the aforesaid polymerase, the aforesaid mono-functional DNA glycosylase, the aforesaid linking nucleotide that serves as a substrate for mono-functional DNA glycosylase, the aforesaid abasic site endonuclease, and the aforesaid 3′ phosphatase activity-possessing enzyme. The kit is used according to the aforesaid method of the present disclosure.

In addition, the present disclosure provides a method of regenerating a reusable initiator for nucleic acid synthesis, which includes:
providing a mono-functional DNA glycosylase as described above;
providing an initiator for nucleic acid synthesis as described above and a synthesized nucleic acid, the synthesized nucleic acid being linked to the initiator right after a linking nucleotide as described above;
subjecting the substrate base to an excision treatment as described above with the mono-functional DNA glycosylase;
providing an abasic site endonuclease as described above;
subjecting the abasic site to a cleavage treatment as described above with the abasic site endonuclease;
providing a 3′ phosphatase activity-possessing enzyme as described above; and
subjecting the 3′-terminal nucleotide of the initiator to a dephosphorylation treatment as described above with the 3′ phosphatase activity-possessing enzyme.

The disclosure will be further described by way of the following examples. However, it should be understood that the following examples are solely intended for the purpose of illustration and should not be construed as limiting the disclosure in practice.

### EXAMPLES

### Example 1. Template-Independent Nucleic Acid Synthesis and Reversion of Synthesis Initiator Back to its Original Form by Virtue of Uracil-DNA Glycosylase (UDG), Endonuclease VIII (Nei), and T4 Polynucleotide Kinase with 3′ Phosphatase Activity (T4 PNKP)

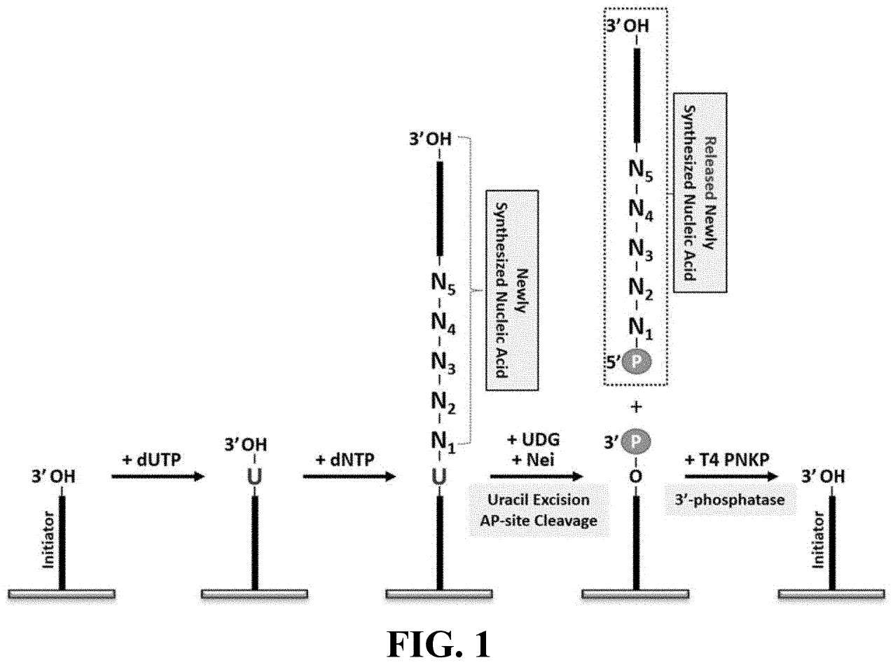

To test whether an initiator used for a template-independent nucleic acid synthesis can be converted back to its original form after nucleic acid synthesis, the following experimental steps were conducted. The detail scheme for the template-independent nucleic acid synthesis using a linking deoxyuridine nucleotide and the reversion of the initiator to its original form utilizing enzymes as applied in this example is illustrated in FIG. 1.

A. Template-Independent Nucleic Acid Synthesis Initiated with the Linking Deoxyuridine Triphosphate (dUTP)

An initiator (a single-stranded 21-mer polynucleotide of SEQ ID NO: 1) with a 5′-hexachloro-fluorescein (HEX) label at the 5′ end thereof and a hydroxyl group at the 3′ terminus thereof was synthesized by Integrated DNA Technologies (Coralville, Iowa, United States). The template-independent nucleic acid synthesis reaction was performed using a 3′ to 5′ exonuclease-deficient Pfu DNA polymerase (Pfu^{exo-}) (200 nM) to incorporate a linking deoxyuridine triphosphate (dUTP) (100 μM) to the 3′ end of the initiator.

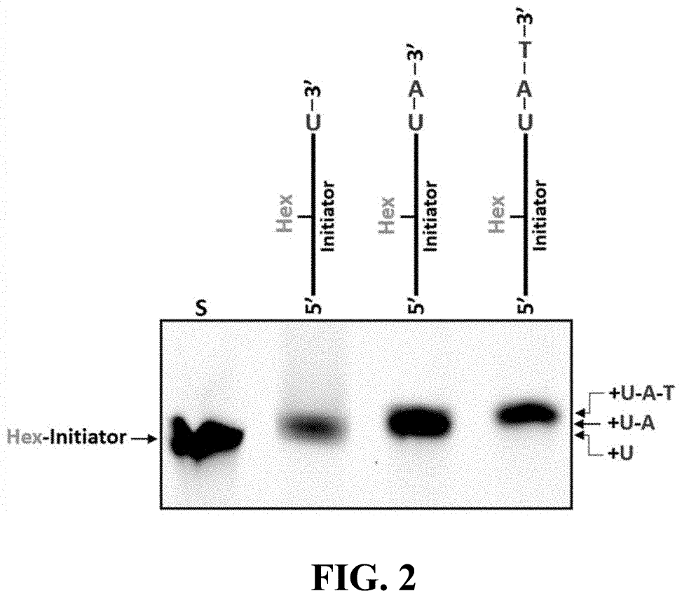

Specifically, the Pfu^{exo-} DNA polymerase (having an amino acid sequence of SEQ ID NO: 8) was prepared as follows. The gene construct encoding an intein-free Pfu DNA polymerase was synthesized by Genomics BioSci and Tech Co. (New Taipei City, Taiwan). The Pfu^{exo-} DNA polymerase was created by changing the Asp¹⁴¹ thereof to Ala (D141A) and the Glu¹⁴³ thereof to Ala (E143A) on the gene backbone using the Q5 Site-directed Mutagenesis Kit from New England Biolabs (Ipswich, Mass., United States). The Pfu^{exo-} DNA polymerase was expressed in E. coli BL21 (DE3) cells and purified through Sepharose-Q and heparin columns using Akta FPLC system from GE Healthcare Life Sciences (Marlborough, Mass., United States). As illustrated in FIG. 2, deoxyuridine monophosphate (dUMP) was efficiently incorporated by the Pfu^{exo-} DNA polymerase into the 3′-end of the initiator.

B. Template-Independent Nucleic Acid Synthesis Right After the Linking dUMP at the 3′ End of the Initiator

To demonstrate the template-independent nucleic acid synthesis right after the linking dUMP at the 3′-end of the synthesis initiator, the Pfu^{exo-} DNA polymerase (200 nM) was used to stepwise incorporate a 3′-O-azidomethyl-dATP and a 3′-O-azidomethyl-dTTP (100 μM) (Jena Bioscience, Erfurt, Germany) to the initiator containing the linking dUMP at the 3′ terminus. The synthesis reaction was initiated by addition of 10 mM manganese cations and then incubated at 75° C. for 30 minutes. The reaction was stopped by adding 10 μL of a 2×quench solution (95% deionized formamide and 25 mM EDTA) and subjected to the heat denaturation at 98° C. for 10 minutes. The reaction products were analyzed by a 15% denaturing urea-polyacrylamide gel, and were visualized by Amersham Typhoon Imager, GE Healthcare Life Sciences (Marlborough, Mass., United States).

As illustrated in FIG. 2, the template-independent nucleic acid synthesis using the Pfu^{exo-} DNA polymerase can incorporate dAMP and dTMP sequentially right after the linking dUMP at the 3′ end of the initiator (the resulting product containing the initiator, the linking dUMP, and dAMP and dTMP has SEQ ID NO: 2). Accordingly, the template-independent nucleic acid synthesis reaction can continue to synthesize a 16-mer polynucleotide of SEQ ID NO: 3 and therefore generate a 38-mer nucleic acid (SEQ ID NO: 4) containing the initiator, the linking dUMP, and the newly synthesized 16-mer polynucleotide.

Please note that since the template-independent nucleic acid synthesis is within the expertise and routine skills of those skilled in the art, the 16-mer nucleic acid of SEQ ID NO: 3 can be synthesized de novo by those skilled in the art with the information provided herein. In this example, to simplify the experimental procedures, the 16-mer nucleic acid was synthesized by Integrated DNA Technologies (Coralville, Iowa, United States), and was linked to the initiator with the linking dUMP as described in section C below to symbolize the template-independent nucleic acid synthesis of the 16-mer nucleic acid.

### C. The Release of Newly Synthesized Nucleic Acid and the Reversion of Synthesis Initiator Back to its Original Form by the Combined Treatments of UDG, Nei, and T4 PNKP

To demonstrate the feasibility of releasing the newly synthesized nucleic acid and regenerating the synthesis initiator by virtue of enzymes, the 38-mer nucleic acid (SEQ ID NO: 4) containing the initiator, the linking dUMP, and the newly synthesized 16-mer polynucleotide was prepared. Specifically, the 16-mer polynucleotide was linked to the initiator with the linking dUMP using the Pfu^{exo-} DNA polymerase.

The 38-mer nucleic acid (25 nM) was subjected to the uracil-excision, the abasic site/nucleic acid backbone cleavage, and the dephosphorylation reaction by the addition of 10 units of UDG, Nei, and T4 PNKP purchased from New England Biolabs (Ipswich, Mass., United States), respectively. The reaction was conducted in the 1×Cleavage Buffer [10 mM MgCl₂, 50 mM KCl, 5 mM dithiothreitol (DTT), and 50 mM Tris-HCl, pH7.5] at 37° C. for 15 minutes. The preparation of such the 38-mer nucleic acid (SEQ ID NO: 4) was confirmed by a 15% denaturing urea-polyacrylamide gel as described above.

In the control experiments, the 38-mer nucleic acid (SEQ ID NO: 4) was also subjected to the treatment with UDG, Nei or the mixture of UDG and Nei under the identical experimental conditions described above. Each reaction was then stopped by adding 10 μL of a 2×quench solution (95% formamide and 25 mM EDTA), and the enzymes were inactivated by heating at 98° C. for 10 minutes. The reaction products were analyzed by a 20% denaturing urea-polyacrylamide gel, and were visualized by Amersham Typhoon Imager, GE Healthcare Life Sciences (Marlborough, Mass., United States).

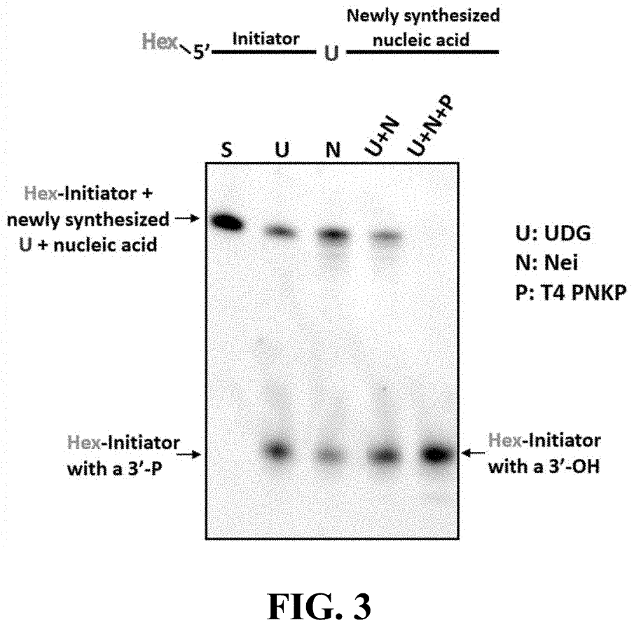

As illustrated in FIG. 3, the treatments with the mixture of UDG, Nei, and T4 PNKP resulted in the removal of the linking dUMP from the single-stranded 38-mer nucleic acid, the release of the newly synthesized 16-mer polynucleotide (SEQ ID NO: 3), and the regeneration of the initiator (SEQ ID NO: 1) with a hydroxyl group at the 3′ terminus. None of UDG alone, Nei alone, or the combination of UDG and Nei can efficiently and completely release the newly synthesized nucleic acid and concurrently regenerate the initiator with the hydroxyl group at the 3′ end.

### Example 2. Template-Independent Nucleic Acid Synthesis and Reversion of Synthesis Initiator to its Original Form by Virtue of Alkyladenine DNA Glycosylase (AAG), Nei, and T4 PNKP

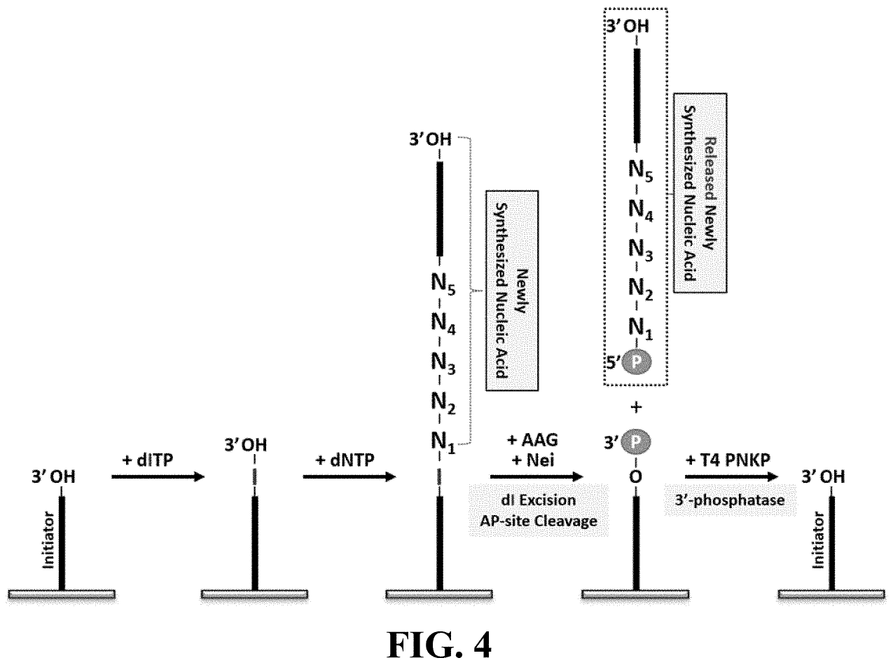

To test whether an initiator used for a template-independent nucleic acid synthesis can be converted back to its original form after nucleic acid synthesis, the following experimental procedures were conducted. The detail scheme for the template-independent nucleic acid synthesis using the linking deoxyinosine triphosphate (dITP) and the reversion of the initiator to its original form utilizing the enzymes as applied in this example is illustrated in FIG. 4.

A. Template-Independent Nucleic Acid Synthesis Initiated with the Linking dITP

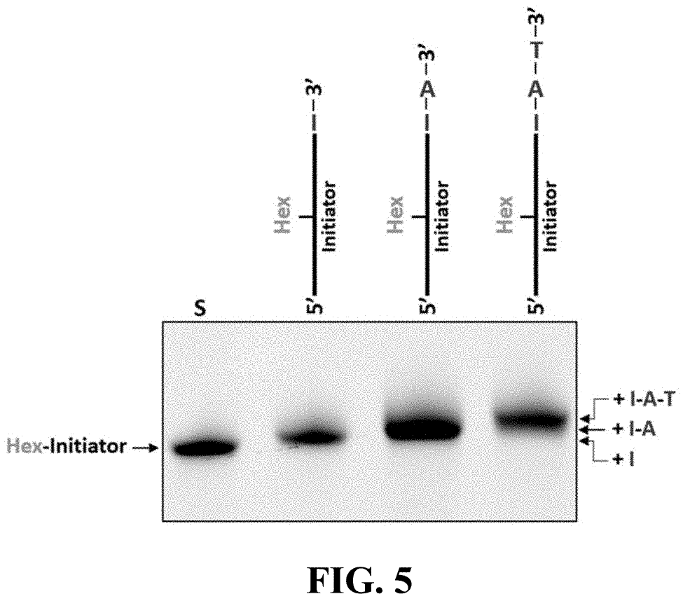

The initiator (SEQ ID NO: 1) with the 5′-hexachloro-fluorescein(HEX) label at the 5′ end and an unprotected hydroxyl group at the 3′ terminus was used. The template-independent nucleic acid synthesis reaction was performed using the Pfu^{exo-} DNA polymerase (200 nM) as described in Example 1 to incorporate a linking dITP (100 μM) to the 3′ end of the initiator. As illustrated in FIG. 5, the deoxyinosine monophosphate (dIMP) was efficiently incorporated by the Pfu^{exo-} DNA polymerase into the 3′-end of the initiator.

B. Template-Independent Nucleic Acid Synthesis Right After the Linking dIMP at the 3′ End of the Initiator

To demonstrate the template-independent nucleic acid synthesis right after the linking dIMP at the 3′-end of the synthesis initiator, the Pfu^{exo-} DNA polymerase (200 nM) was used to stepwise incorporate a 3′-O-azidomethyl-dATP and a 3′-O-azidomethyl-dTTP (100 μM) (Jena Bioscience, Erfurt, Germany) to the initiator containing the linking dIMP at the 3′ terminus. The synthesis reaction was initiated by addition of 10 mM manganese cations and then incubated at 75° C. for 30 minutes. The reaction was stopped by adding 10 μL of a 2×quench solution (95% deionized formamide and 25 mM EDTA) and subjected to the heat denaturation at 98° C. for 10 minutes. The reaction products were analyzed by a 15% denaturing urea-polyacrylamide gel, and were visualized by Amersham Typhoon Imager, GE Healthcare Life Sciences (Marlborough, Mass., United States).

As illustrated in FIG. 5, the template-independent nucleic acid synthesis using the Pfu^{exo-} DNA polymerase can incorporate dAMP and dTMP sequentially right after the linking dIMP at the 3′ end of the initiator (the resulting product containing the initiator, the linking dIMP, and dAMP and dTMP has SEQ ID NO: 5). Accordingly, the template-independent nucleic acid synthesis reaction can continue to synthesize the 16-mer polynucleotide of SEQ ID NO: 3 and generate a 38-mer nucleic acid (SEQ ID NO: 6) containing the initiator, the linking dIMP, and the newly synthesized 16-mer polynucleotide. The preparation of such a 38-mer nucleic acid (SEQ ID NO: 6) was confirmed by a 15% denaturing urea-polyacrylamide gel as described above.

Please note that since the template-independent nucleic acid synthesis is within the expertise and routine skills of those skilled in the art, the 16-mer nucleic acid of SEQ ID NO: 3 can be synthesized de novo by those skilled in the art with the information provided herein. In this example, to simplify the experimental procedures, the 16-mer nucleic acid was linked to the initiator with the linking dIMP as described in section C below to symbolize the template-independent nucleic acid synthesis of the 16-mer nucleic acid.

### C. The Release of Newly Synthesized Nucleic Acid and the Reversion of Synthesis Initiator Back to its Original Form by the Combined Treatments of AAG, Nei, and T4 PNKP

To demonstrate the feasibility of releasing the newly synthesized nucleic acid and regenerating the synthesis initiator by virtue of enzymes, the single-stranded 38-mer nucleic acid (SEQ ID NO: 6) containing the initiator (SEQ ID NO: 1), the linking dIMP, and the newly synthesized 16-mer polynucleotide (SEQ ID NO: 3) was prepared. Specifically, the 16-mer polynucleotide was linked to the initiator with the linking dIMP using the Pfu^{exo-} DNA polymerase.

The single-stranded 38-mer nucleic acid (25 nM) was subjected to the inosine-excision, the abasic site/nucleic acid backbone cleavage, and the dephosphorylation reaction by the addition of 10 units of AAG, Nei, and T4 PNKP purchased from New England Biolabs (Ipswich, Mass., United States), respectively. The reaction was conducted in a 1×Cleavage Buffer [10 mM MgCl₂, 50 mM KCl, 5 mM dithiothreitol (DTT), and 50 mM Tris-HCl; pH 7.5] at 37° C. for 15 minutes.

In the control experiments, the single-stranded 38-mer nucleic acid (SEQ ID NO: 6) was also subjected to the treatment with UDG, Nei or the mixture of AAG and Nei under the identical experimental conditions. Each reaction was then stopped by adding 10 μL of a 2×quench solution (95% formamide and 25 mM EDTA), and the enzymes were inactivated by heating at 98° C. for 10 minutes. The reaction products were analyzed by a 20% denaturing urea-polyacrylamide gel, and were visualized by Amersham Typhoon Imager, GE Healthcare Life Sciences (Marlborough, Mass., United States).

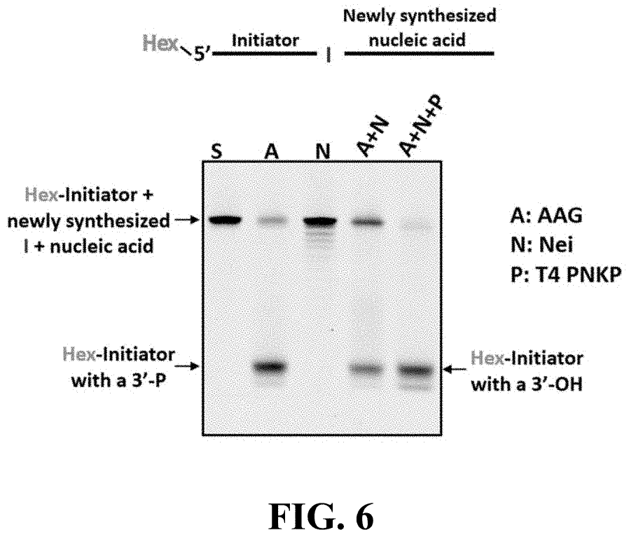

As illustrated in FIG. 6, the treatments with the mixture of AAG, Nei, and T4 PNKP resulted in the removal of the linking dIMP from the single-stranded 38-mer nucleic acid, the release of the newly synthesized 16-mer polynucleotide (SEQ ID NO: 3), and the regeneration of the initiator (SEQ ID NO: 1) with a hydroxyl group at the 3′ terminus. None of AAG alone, Nei alone, or the combination of AAG and Nei can efficiently and completely cut off the newly synthesized nucleic acid and concurrently regenerate the initiator with the hydroxyl group at the 3′ end.

### Example 3. Template-Dependent Nucleic Acid Synthesis and Reversion of Synthesis Initiator Back to its Original Form by Virtue of UDG, Nei, and T4 PNKP

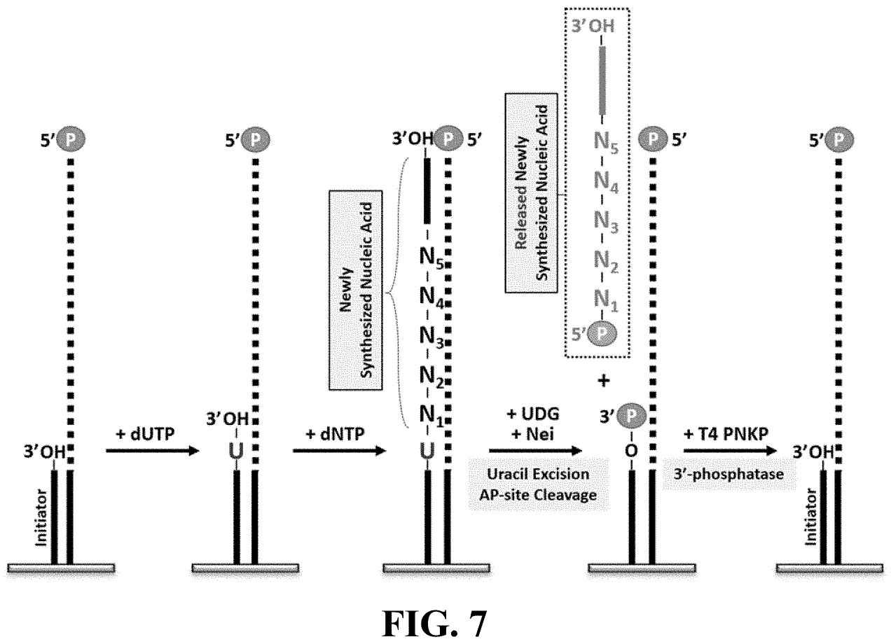

To test whether an initiator used for a template-dependent nucleic acid synthesis can be converted back to its original form after nucleic acid synthesis, the following experimental procedures were conducted. The detail scheme for the template-dependent nucleic acid synthesis using the linking dUTP and the reversion of the initiator to its original form utilizing the enzymes as applied in this example is illustrated in FIG. 7.

### A. The Release of Newly Synthesized Nucleic Acid and the Reversion of Synthesis Initiator Back to its Original Form by the Combined Treatments of UDG, Nei, and T4 PNKP

To demonstrate the feasibility of releasing the newly synthesized nucleic acid and regenerating the synthesis initiator by virtue of enzymes, the single-stranded 38-mer nucleic acid (SEQ ID NO: 4) containing the initiator, the linking dUMP, and the newly synthesized 16-mer polynucleotide was prepared as described in Example 1. To exemplify the template-dependent nucleic acid synthesis, the single-stranded 38-mer nucleic acid (SEQ ID NO: 4) was hybridized with a complementary single-stranded 38-mer nucleic acid (SEQ ID NO: 7) by heating at 95° C. for 10 minutes, followed by slowly cooling down to 4° C. to form a duplex, blunt-end, double stranded 38-mer nucleic acid. The complementary single-stranded 38-mer nucleic acid (SEQ ID NO: 7) was obtained from Integrated DNA Technologies (Coralville, Iowa, United States).

25 nM of the duplex 38-mer nucleic acid was subjected to the uracil-excision, the abasic site/nucleic acid backbone cleavage, and the dephosphorylation reaction by the addition of 10 units of UDG, Nei, and T4 PNKP purchased from New England Biolabs (Ipswich, Mass., United States), respectively. The reaction was conducted in a 1×Cleavage Buffer [10 mM MgCl₂, 50 mM KCl, 5 mM dithiothreitol (DTT), and 50 mM Tris-HCl; pH 7.5] at 37° C. for 15 minutes.

In the control experiments, the duplex 38-mer nucleic acid was subjected to the treatment with UDG, Nei or the mixture of UDG and Nei under the identical experimental conditions. Each reaction was then stopped by adding 10 μL of a 2×quench solution (95% formamide and 25 mMEDTA), the enzymes were inactivated, and the duplex 38-mer nucleic acid was denatured by heating at 98° C. for 10 minutes. The reaction products were analyzed by a 20% denaturing urea-polyacrylamide gel, and were visualized by Amersham Typhoon Imager, GE Healthcare Life Sciences (Marlborough, Mass., United States).

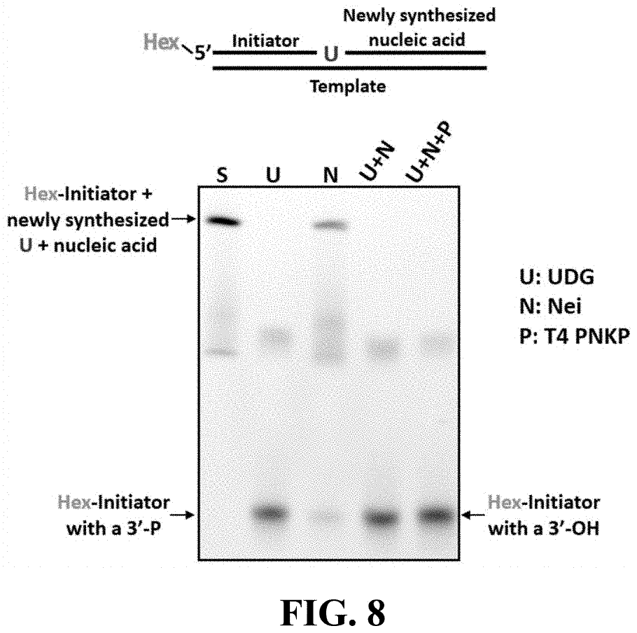

As shown in FIG. 8, the treatments with the mixture of UDG, Nei, and T4 PNKP resulted in the removal of the linking dUMP from the 38-mer nucleic acid, the release of the newly synthesized 16-mer polynucleotide (SEQ ID NO: 3) after the heat denaturation of the duplex 38-mer nucleic acid, and the regeneration of the initiator (SEQ ID NO: 1) with the hydroxyl group at the 3′ terminus. None of UDG alone, Nei alone, or the combination of UDG and Nei can efficiently and completely release the newly synthesized nucleic acid after the heat denaturation of the duplex 38-mer nucleic acid and concurrently regenerate the initiator with the hydroxyl group at the 3′ end.

### Example 4. Template-Dependent Nucleic Acid Synthesis and Reversion of Synthesis Initiator Back to its Original Form by Virtue of AAG, Nei, and T4 PNKP

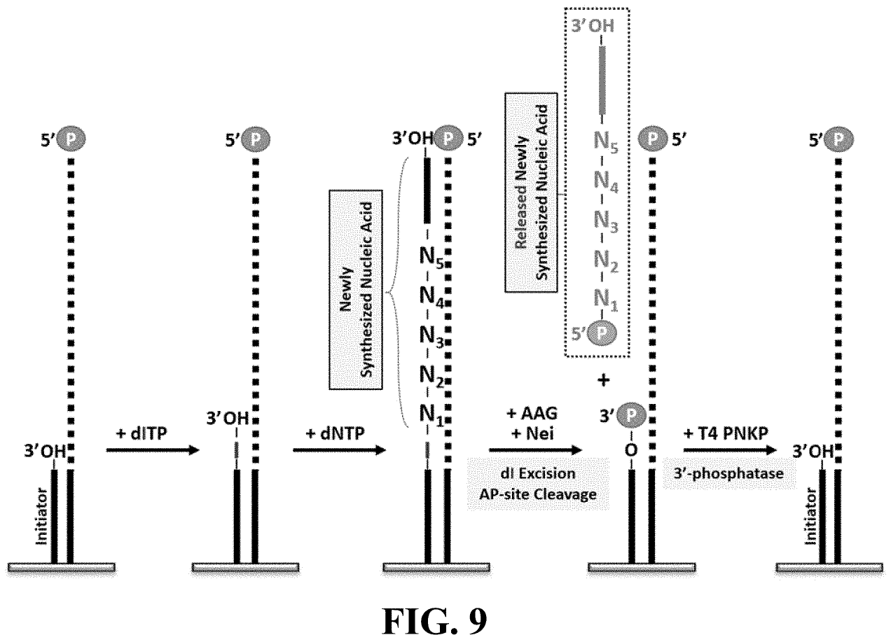

To test whether an initiator used for a template-dependent nucleic acid synthesis can be converted back to its original form after nucleic acid synthesis, the following experimental procedures were conducted. The detail scheme for the template-dependent nucleic acid synthesis using the linking dITP and the reversion of the initiator to its original form utilizing the enzymes as applied in this example is illustrated in FIG. 9.

### A. The Release of Newly Synthesized Nucleic Acid and the Reversion of Synthesis Initiator Back to its Original Form by the Combined Treatments of AAG, Nei, and T4 PNKP

To demonstrate the feasibility of releasing the newly synthesized nucleic acid and regenerating the synthesis initiator by virtue of enzymes, the single-stranded 38-mer nucleic acid (SEQ ID NO: 6), containing the initiator, the linking dIMP, and the newly synthesized 16-mer polynucleotide was prepared as described in Example 2. To exemplify the template-dependent nucleic acid synthesis, the single-stranded 38-mer nucleic acid (SEQ ID NO: 6) was hybridized with the complementary 38-mer nucleic acid (SEQ ID NO: 7) by heating at 95° C. for 10 minutes, followed by slowly cooling down to 4° C. to form a duplex, blunt-end, double stranded 38-mer nucleic acid. The complementary single-stranded 38-mer nucleic acid (SEQ ID NO: 7) was obtained from Integrated DNA Technologies (Coralville, Iowa, United States). 25 nM of the duplex 38-mer nucleic acid was subjected to the inosine-excision, the abasic site/nucleic acid backbone cleavage, and the dephosphorylation reaction by the addition of 10 units of AAG, Nei, and T4 PNKP purchased from New England Biolabs (Ipswich, Mass., United States), respectively. The reaction was conducted in a 1×Cleavage Buffer [10 mM MgCl₂, 50 mM KCl, 5 mM dithiothreitol (DTT), and 50 mM Tris-HCl; pH 7.5] at 37° C. for 15 minutes.

In the control experiments, the duplex 38-mer nucleic acid was subjected to the treatment with AAG, Nei or the mixture of AAG and Nei under the identical experimental conditions. Each reaction was then stopped by adding 10 μL of a 2×quench solution (95% formamide and 25 mM EDTA), the enzymes were inactivated, and the duplex nucleic acid was denatured by heating at 98° C. for 10 minutes. The reaction products were analyzed by a 20% denaturing urea-polyacrylamide gel, and were visualized by Amersham Typhoon Imager, GE Healthcare Life Sciences (Marlborough, Mass., United States).

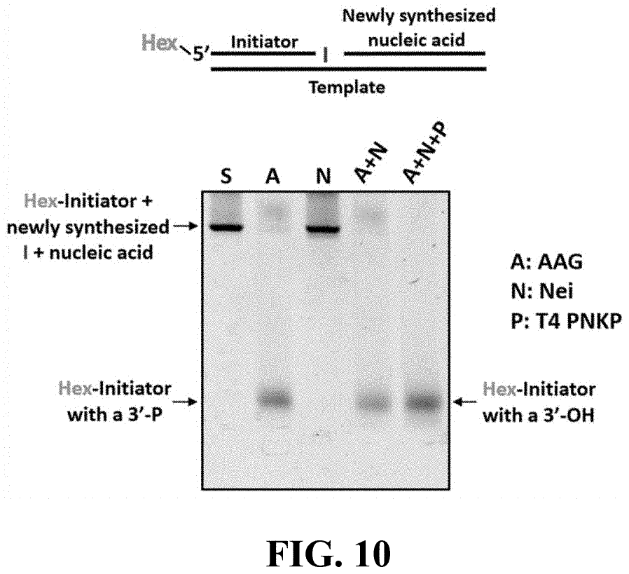

As shown in FIG. 10, the treatments with the mixture of AAG, Nei, and T4 PNKP resulted in the removal of the linking dIMP from the duplex 38-mer nucleic acid, the release of the newly synthesized 16-mer polynucleotide (SEQ ID NO: 3) after the heat denaturation of the duplex 38-mer nucleic acid, and the regeneration of the initiator (SEQ ID NO: 1) with the hydroxyl group at the 3′ terminus. None of AAG alone, Nei alone, or the combination of AAG and Nei can efficiently and completely release the newly synthesized nucleic acid after the heat denaturation of the duplex 38-mer nucleic acid and concurrently regenerate the initiator with the hydroxyl group at the 3′ end.

All patents and references cited in this specification are incorporated herein in their entirety as reference. Where there is conflict, the descriptions in this case, including the definitions, shall prevail.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. A method for nucleic acid synthesis and regeneration of a reusable initiator for such synthesis, comprising:
exposing an initiator attached to a solid support for nucleic acid synthesis to a linking nucleotide in the presence of a polymerase so that the linking nucleotide is incorporated to the initiator, the linking nucleotide having a substrate base, a substrate sugar, and a 3′ hydroxyl group;
exposing the initiator containing the linking nucleotide to nucleotide monomers in the presence of the polymerase, so that a nucleic acid is synthesized and is coupled to the initiator right after the linking nucleotide;
providing a mono-functional DNA glycosylase, the linking nucleotide with the substrate base being recognizable and excisable by the mono-functional DNA glycosylase;
subjecting the substrate base to an excision treatment with the mono-functional DNA glycosylase, so that the substrate base is excised by the mono-functional DNA glycosylase to generate an abasic site;
providing an abasic site endonuclease, the resulting abasic site being recognizable and the substrate sugar being cleavable by the abasic site endonuclease;
subjecting the abasic site to a cleavage treatment with the abasic site endonuclease, so that the substrate sugar and the backbone of the nucleic acid at the abasic site are both cleaved to release the nucleic acid from the initiator, so that a 3′-terminal nucleotide of the initiator has a 3′ phosphate group, and so that a 5′-terminal nucleotide of the synthesized nucleic acid has a 5′ phosphate group;
providing a 3′ phosphatase activity-possessing enzyme; and
subjecting the 3′-terminal nucleotide of the initiator to a dephosphorylation treatment with the 3′ phosphatase activity-possessing enzyme, so that the 3′ phosphate group of the 3′-terminal nucleotide of the initiator is converted back to the original 3′ hydroxyl group.

2. The method according to claim 1, wherein the mono-functional DNA glycosylase is selected from the group consisting of uracil-DNA glycosylase, alkyladenine DNA glycosylase, single-strand-selective monofunctional uracil DNA glycosylase 1, methyl-binding domain glycosylase 4, thymine DNA glycosylase, mutY homolog DNA glycosylase, alkylpurine glycosylase C, alkylpurine glycosylase D, 8-oxo-guanine glycosylase 1 without abasic site lyase activity, endonuclease III-like 1 without abasic site lyase activity, endonuclease VIII-like glycosylase 1 without abasic site lyase activity, endonuclease VIII-like glycosylase 2 without abasic site lyase activity, endonuclease VIII-like glycosylase 3 without abasic site lyase activity, and enzymatically active fragments thereof.

3. The method according to claim 2, wherein the mono-functional DNA glycosylase is one of uracil-DNA glycosylase and alkyladenine DNA glycosylase.

4. The method according to claim 1, wherein the abasic site endonuclease is selected from the group consisting of endonuclease VIII, endonuclease III, and enzymatically active fragments thereof.

5. The method according to claim 4, wherein the abasic site endonuclease is endonuclease VIII.

6. The method according to claim 1, wherein the 3′ phosphatase activity-possessing enzyme is selected from the group consisting of a polynucleotide kinase 3′-phosphatase, a 3′-phosphoesterase, and enzymatically active fragments thereof.

7. The method according to claim 6, wherein the 3′ phosphatase activity-possessing enzyme is selected from the group consisting of T4 polynucleotide kinase with 3′phosphatase activity and zinc finger DNA 3′-phosphoesterase.

8. The method according to claim 1, wherein the substrate base of the linking nucleotide is selected from the group consisting of uracil, hypoxanthine, thymine, cytosine, guanine, 5-fluorouracil, 5-hydroxymethyluracil, 5-formylcytosine, 5-carboxylcytosine, 3-methyladenine, 3-methylguanine, 7-methyladenine, 7-methylguanine, N⁶-methyladenine, 8-oxo-7,8-dihydroguanine, 5-hydroxyl cytosine, 5-hydroxyl uracil, dihydroxyuracil, ethenocytosine, ethenoadenine, thymine glycol, cytosine glycol, 2,6-diamino-4-hydroxy-5-N-methylformamidopyrimidine, a formamidopyrimidine derivative of adenine, a formamidopyrimidine derivative of guanine, adenine opposite guanine, uracil opposite guanine, uracil opposite adenine, thymine opposite guanine, ethenocytosine opposite guanine, adenine opposite 8-oxo-7,8-dihydroguanine, and 2-hydroxyladenine opposite guanine.

9. The method according to claim 8, wherein the substrate base of the linking nucleotide is one of uracil and hypoxanthine.

10. The method according to claim 1, wherein the initiator, the synthesized nucleic acid, and the linking nucleotide are each in one of a template-independent form and a template-dependent form.

11. The method according to claim 1, wherein the polymerase is selected from the group consisting of a family-A DNA polymerase, a family-B DNA polymerase, a family-C DNA polymerase, a family-D DNA polymerase, a family-X DNA polymerase, a family-Y DNA polymerase, a reverse transcriptase, and enzymatically active fragments thereof.

12. A kit for nucleic acid synthesis and regeneration of a reusable nucleic acid for such synthesis, comprising:
a polymerase and a linking nucleotide for nucleic acid synthesis;
a mono-functional DNA glycosylase;
an abasic site endonuclease; and
a 3′ phosphatase activity-possessing enzyme;
wherein the kit is used according to a method as described in claim 1.

13. A method of regenerating a reusable initiator for nucleic acid synthesis, comprising:
providing a mono-functional DNA glycosylase;
providing an initiator for nucleic acid synthesis and a synthesized nucleic acid, the initiator being attached to a solid support, the synthesized nucleic acid being linked to the initiator right after a linking nucleotide having a substrate base and a substrate sugar, the linking nucleotide with the substrate base being recognizable and excisable by the mono-functional DNA glycosylase;
subjecting the substrate base to an excision treatment with the mono-functional DNA glycosylase, so that the substrate base is excised by the mono-functional DNA glycosylase to generate an abasic site;
providing an abasic site endonuclease, the resulting abasic site being recognizable and the substrate sugar being cleavable by the abasic site endonuclease;
subjecting the abasic site to a cleavage treatment with the abasic site endonuclease, so that the substrate sugar and the backbone of the nucleic acid at the abasic site are both cleaved to release the synthesized nucleic acid from the initiator, so that a 3′-terminal nucleotide of the initiator has a 3′ phosphate group, and so that a 5′-terminal nucleotide of the synthesized nucleic acid has a 5′ phosphate group;
providing a 3′ phosphatase activity-possessing enzyme; and
subjecting the 3′-terminal nucleotide of the initiator to a dephosphorylation treatment with the 3′ phosphatase activity-possessing enzyme, so that the 3′ phosphate group of the 3′-terminal nucleotide of the initiator is converted back to an original 3′ hydroxyl group.

14. The method according to claim 13, wherein the mono-functional DNA glycosylase is selected from the group consisting of uracil-DNA glycosylase, alkyladenine DNA glycosylase, single-strand-selective monofunctional uracil DNA glycosylase 1, methyl-binding domain glycosylase 4, thymine DNA glycosylase, mutY homolog DNA glycosylase, alkylpurine glycosylase C, alkylpurine glycosylase D, 8-oxo-guanine glycosylase 1 without abasic site lyase activity, endonuclease III-like 1 without abasic site lyase activity, endonuclease VIII-like glycosylase 1 without abasic site lyase activity, endonuclease VIII-like glycosylase 2 without abasic site lyase activity, endonuclease VIII-like glycosylase 3 without abasic site lyase activity, and enzymatically active fragments thereof.

15. The method according to claim 14, wherein the mono-functional DNA glycosylase is one of uracil-DNA glycosylase and alkyladenine DNA glycosylase.

13. method according to claim 13, wherein the abasic site endonuclease is selected from the group consisting of endonuclease VIII, endonuclease III, and enzymatically active fragments thereof.

17. The method according to claim 16, wherein the abasic site endonuclease is endonuclease VIII.

18. The method according to claim 13, wherein the 3′ phosphatase activity-possessing enzyme is selected from the group consisting of a polynucleotide kinase 3′-phosphatase, a 3′-phosphoesterase, and enzymatically active fragments thereof.

19. The method according to claim 18, wherein the 3′ phosphatase activity-possessing enzyme is selected from the group consisting of T4 polynucleotide kinase with 3′phosphatase activity and zinc finger DNA 3′-phosphoesterase.

20. The method according to claim 13, wherein the substrate base of the linking nucleotide is selected from the group consisting of uracil, hypoxanthine, thymine, cytosine, guanine, 5-fluorouracil, 5-hydroxymethyluracil, 5-formylcytosine, 5-carboxylcytosine, 3-methyladenine, 3-methylguanine, 7-methyladenine, 7-methylguanine, N⁶-methyladenine, 8-oxo-7,8-dihydroguanine, 5-hydroxyl cytosine, 5-hydroxyl uracil, dihydroxyuracil, ethenocytosine, ethenoadenine, thymine glycol, cytosine glycol, 2,6-diamino-4-hydroxy-5-N-methylformamidopyrimidine, a formamidopyrimidine derivative of adenine, a formamidopyrimidine derivative of guanine, adenine opposite guanine, uracil opposite guanine, uracil opposite adenine, thymine opposite guanine, ethenocytosine opposite guanine, adenine opposite 8-oxo-7,8-dihydroguanine, and 2-hydroxyladenine opposite guanine.

21. The method according to claim 13, wherein the initiator, the synthesized nucleic acid, and the linking nucleotide are each in one of a template-independent form and a template-dependent form.
